(19) United States
(12) Patent Application Publication
(10) Pub. No.: US 2019/0076027 A1
(43) Pub. Date: Mar. 14, 2019

(54) PROCESSING APPARATUS AND PROCESSING METHOD CONCERNING MEDICAL IMAGES

(71) Applicant: CANON KABUSHIKI KAISHA, Tokyo (JP)

(72) Inventor: Ryuichi Nanaumi, Tokyo (JP)

(21) Appl. No.: 16/123,523

(22) Filed: Sep. 6, 2018

(30) Foreign Application Priority Data
Sep. 14, 2017 (JP) 2017-176982

(51) Int. Cl. A61B 5/00; G06T 7/00; G06T 5/00

(52) U.S. Cl. CPC A61B 5/0095; G06T 7/0014; G06T 5/003; A61B 2576/00; A61B 5/7425; G06T 2207/20208; G06T 5/009

(57) ABSTRACT

A processing apparatus concerning medical images includes a reference image readout unit configured to read out a reference image acquired by photoacoustic imaging, a target image readout unit configured to read out a target image for diagnostics acquired by photoacoustic imaging, and a calculation unit configured to modify at least one of a read-out reference image and a read-out target image, wherein the reference image and the target image are read out while being associated with meta-information, and wherein the calculation unit modifies at least one of the read-out reference image and the read-out target image based on the meta-information.

### BACKGROUND OF THE INVENTION

### Field of the Invention

Aspects of the present disclosure generally relate to a processing apparatus and a processing method concerning medical images.

### Description of the Related Art

As a technology for acquiring information about the inside of a subject, such as a biological object, by receiving acoustic waves, subject information acquisition apparatuses, such as a photoacoustic imaging apparatus and an ultrasonic echo imaging apparatus, have been heretofore proposed.

For example, the photoacoustic imaging apparatus has been proved to be of usefulness in the diagnosis of, especially, skin cancer and breast cancer. The photoacoustic imaging apparatus is increasingly expected to become medical equipment serving as an alternative to, for example, an ultrasonic echo diagnosis apparatus, an X-ray apparatus, and a magnetic resonance imaging (MRI) apparatus, which have conventionally been used for the above diagnosis.

When a biological tissue is irradiated with measurement light, such as visible light or near-infrared light, a light-absorbing material inside the biological object, for example, a material such as hemoglobin in the blood, absorbs energy included in the measurement light and thus instantaneously expands and, as a result, acoustic waves are generated. This phenomenon is called a photoacoustic effect, and the generated acoustic waves are also called photoacoustic waves.

The photoacoustic imaging apparatus measures such photoacoustic waves to visualize information about a biological tissue. The technology of tomography using the photoacoustic effect is also called photoacoustic imaging (PAI).

Moreover, in the field of medical images, a technique for aiding diagnosis by comparing a target image (an image read for interpretation) with a reference image has been proposed. Japanese Patent Application Laid-Open No. 2017-000675 discusses a processing apparatus concerning medical images, which is capable of increasing the accuracy of diagnosis performed with comparison by modifying an image based on images including luminance values or spatial frequency distributions to bring the image qualities of the target image and the reference image close to each other.

However, the processing apparatus discussed in Japanese Patent Application Laid-Open No. 2017-000675 performs modification to the reference image based on only the acquired image, and is, therefore, unlikely to bring the image quality of the reference image sufficiently close to the image quality of the target image.

### SUMMARY OF THE INVENTION

The present disclosure has been accomplished based on the recognition of such an issue. Aspects of the present disclosure are generally directed to a processing apparatus concerning medical images which is capable of performing diagnosis using a comparison between read information and reference information with a higher degree of accuracy.

According to an aspect of the present disclosure, a processing apparatus concerning medical images includes a reference image readout unit configured to read out a reference image acquired by photoacoustic imaging, a target image readout unit configured to read out a target image for diagnostics acquired by photoacoustic imaging, and a calculation unit configured to modify at least one of a read-out reference image and a read-out target image, wherein the reference image and the target image are read out while being associated with meta-information, and wherein the calculation unit modifies at least one of the read-out reference image and the read-out target image based on the meta-information.

According to another aspect of the present disclosure, a processing method concerning medical images includes reading out a reference image acquired by photoacoustic imaging, reading out a target image for diagnostics acquired by photoacoustic imaging, and modifying at least one of a read-out reference image and a read-out target image, wherein the reference image and the target image are read out while being associated with meta-information, and wherein the at least one of the read-out reference image the read-out target image is modified based on the meta-information.

Further features will become apparent from the following description of exemplary embodiments with reference to the attached drawings.

### BRIEF DESCRIPTION OF THE DRAWINGS

FIG. 1 is a schematic diagram of a processing apparatus concerning medical images according to a first exemplary embodiment.

FIG. 2 is a schematic diagram illustrating the processing apparatus concerning medical images and the details of surrounding sections thereof according to the first exemplary embodiment.

FIG. 3 is a flowchart of a processing method concerning medical images according to the first exemplary embodiment.

FIG. 4 is a schematic diagram illustrating the details of a graphical user interface (GUI) which is displayed on a display device.

FIG. 5 is a flowchart of a processing method concerning medical images including a determination of whether to perform modification processing.

FIG. 6 is a schematic diagram of a processing apparatus concerning medical images according to a second exemplary embodiment.

FIG. 7 is a flowchart of a processing method concerning medical images according to the second exemplary embodiment.

FIGS. 8A and 8B are schematic diagrams illustrating statistical values of background optical coefficients.

FIGS. 9A and 9B are schematic diagrams illustrating background optical coefficients of a target image and a reference image.

### DESCRIPTION OF THE EMBODIMENTS

Hereinafter, various exemplary embodiments, features, and aspects of the present disclosure will be described in detail below with reference to the drawings. In this regard, however, for example, the dimension, material, shape, and relative disposition of each constituent component described below can be changed or altered as appropriate according to the configuration of an apparatus to which the present disclosure is applied and various conditions thereof. Therefore, the scope of the present invention is not intended to be limited to the following description.

The present disclosure is related to a technique for processing subject information generated by photoacoustic imaging Therefore, an aspect of the present disclosure includes a processing apparatus concerning medical images or a control method therefor, or an acquisition method, signal processing method, or image processing method for medical images. An aspect of the present disclosure also includes a program that causes an information processing apparatus including hardware resources such as a central processing unit (CPU) to perform the above methods or a storage medium storing such a program. Furthermore, subject information is acquired by an apparatus using a photoacoustic tomography technology, which irradiates a subject with light (electromagnetic waves) and receives (detects) acoustic waves generated at and propagated from a specific position in the subject or on the surface of the subject according to the photoacoustic effect. Read information and reference information cited in the present disclosure are parts of subject information and include, for example, the position of a generation source of acoustic waves generated by light irradiation, an initial sound pressure in the subject, light energy absorption density distributions or absorption coefficients derived from the initial sound pressure, and the density of a material constituting a subject tissue. Specifically, the density of a material constituting a subject tissue includes, for example, a blood component physical property value, such as oxygenated and reduced hemoglobin concentrations or oxygen saturations obtained from those, and the concentrations of fat, collagen, and water. These can be obtained not only as numerical data but also as distribution information associated with each position in the subject. Such distribution information serves as a target image for diagnostics (hereinafter, referred to merely as target image) and a reference image.

The acoustic waves cited in the present specification are typically ultrasonic waves, and include elastic waves called sound waves or acoustic waves. Acoustic waves generated by the photoacoustic effect are referred to as “photoacoustic waves” or “photoultrasonic waves”.

Hereinafter, a first exemplary embodiment of the present disclosure is described in detail with reference to the drawings. The present embodiment is configured to modify a difference in resolution which is dependent on positions in a target image and a reference image. Furthermore, in principle, the same constituent elements are assigned the respective same reference characters, and the description thereof is not repeated.

### <Configuration of a Processing Apparatus Concerning Medical Images>

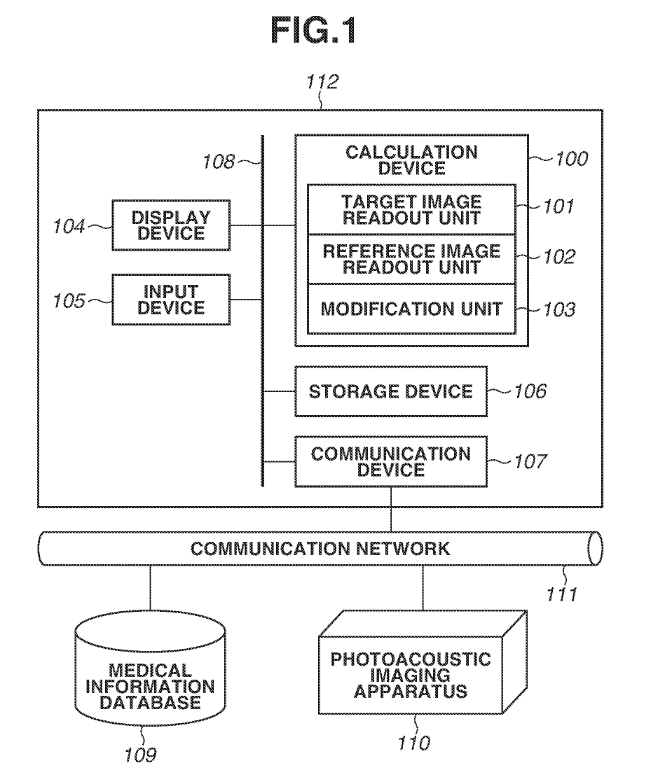

FIG. 1 is a schematic diagram of a processing apparatus 112 concerning medical images according to the present exemplary embodiment. Each constituent element of the processing apparatus 112 concerning medical images is described as follows. The processing apparatus 112 concerning medical images includes a calculation device 100, a display device 104, an input device 105, a storage device 106, a communication device 107, a bus 108, a medical information database 109, a photoacoustic imaging apparatus 110, and a communication network 111. The calculation device 100 includes a target image readout unit 101, a reference image readout unit 102, and a modification unit 103.

Hereinafter, details of each constituent element of the processing apparatus 112 concerning medical images according to the present exemplary embodiment are described.

The calculation device 100 controls an operation of each constituent element of the processing apparatus 112 concerning medical images via the bus 108. Moreover, the calculation device 100 reads out a program, in which a processing method concerning medical images described below is described, stored in the storage device 106, thus causing processing concerning medical images to be performed. The calculation device 100 is typically configured with an element, such as a central processing unit (CPU), a graphics processing unit (GPU), or an analog-to-digital (A/D) converter, or a circuit, such as a field-programmable gate array (FPGA) or an application specific integrated circuit (ASIC). The calculation device 100 is not only configured with one element or circuit but also can be configured with a plurality of elements or circuits. Each processing which is performed by the processing method concerning medical images can be performed by any element or circuit. Moreover, it is favorable that the calculation device 100 is configured to be able to concurrently perform pipeline processing on a plurality of signals. This enables reducing calculation time. Furthermore, although not illustrated in FIG. 1, the calculation device 100 can contain a temporary storage device such as a random access memory (RAM). For example, programs, target images, reference meta-information, images in the process of being processed, and images obtained after being processed, which are stored in the storage device 106, can be read out from or written into the RAM.

Here, meta-information which is cited in the present specification is described. The meta-information is information which, when a photoacoustic imaging apparatus records a captured image on a storage medium, is recorded while being associated with the captured image and which describes the attribute of the captured image. The meta-information includes information about, for example, an image capturing date and time, an image capturing condition, a subject, an imaging apparatus, and a medical institution. The meta-information is recorded while being accompanied by a captured image and is, therefore, also called accompanying information. The meta-information is also called attribute information.

A captured image with its meta-information accompanied thereby serves as a data element included in a database which is classified by, for example, a time series, subjects, and pathological conditions. A medical information database including meta-information presents, to a reader for interpretation or a doctor, an opportunity for comprehensively understanding the pathological condition of a patient based on a group of analysis results obtained by a plurality of different modalities.

The target image readout unit 101 transfers a target image targeted for diagnosis from the medical information database 109 to the storage device 106. During such transfer, the target image readout unit 101 also transfers, in addition to a target image targeted for diagnosis, target image meta-information accompanied thereby (hereinafter referred to as read meta-information). The target image readout unit is also referred to as a read information readout unit.

The reference image readout unit 102 transfers a reference image for aiding diagnosis from the medical information database 109 to the storage device 106. At this time, the reference image readout unit 102 also transfers, in addition to a reference image, reference image meta-information accompanied thereby (hereinafter referred to as reference meta-information). The reference image readout unit is also referred to as a reference information readout unit.

The target image readout unit 101 and the reference image readout unit 102 are typically a program which is stored in the storage device 106 and which is read out and executed by the calculation device 100 at the time of execution of processing concerning medical images, and operate as a part of the calculation device 100.

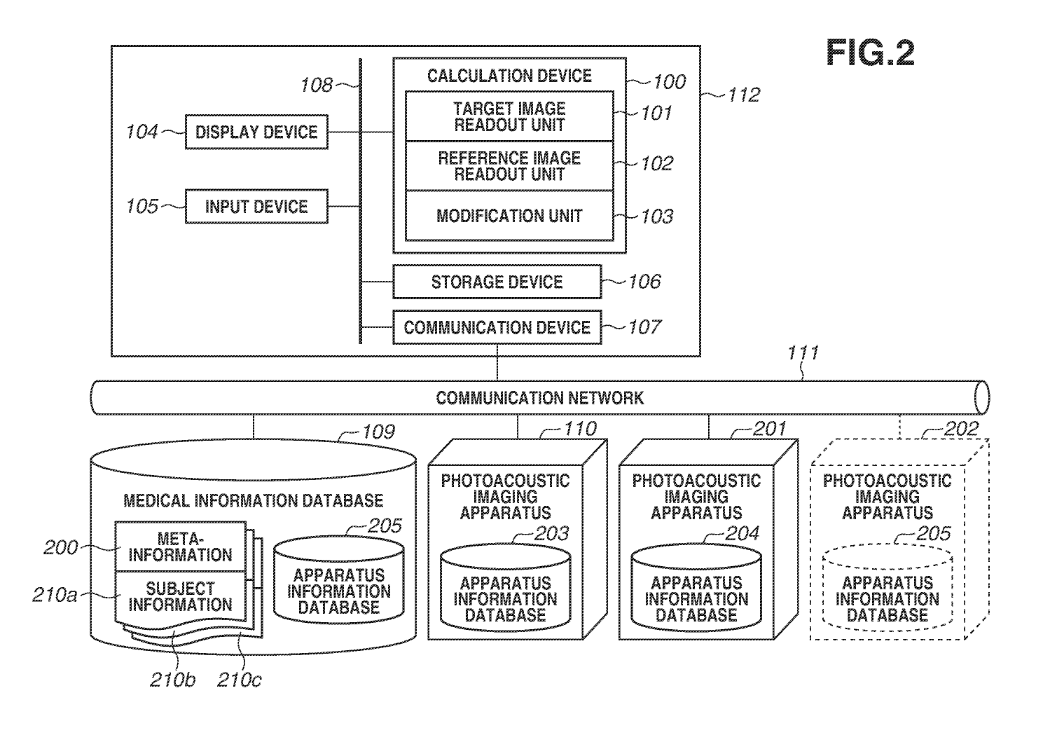

The read meta-information and the reference meta-information are typically recorded concurrently with a corresponding image and associated with the corresponding image as indicated by meta-information 200 illustrated in FIG. 2. The read meta-information and the reference meta-information can be recorded, for example, in a tag format of Digital Imaging and Communications in Medicine (DICOM), which is a general-purpose format for medical images. The read meta-information and the reference meta-information can be saved as a different file associated with an image file. However, meta-information and a corresponding image do not necessarily need to be concurrently recorded on the same storage device, and meta-information in common between a plurality of images can be previously saved on a storage medium different from a storage destination of images such as the medical information database 109 or the photoacoustic imaging apparatus 110.

For example, information specific for the photoacoustic imaging apparatus 110 is described as an example. In this case, as illustrated in FIG. 2, medical information databases (203 and 204) are respectively provided for individual photoacoustic imaging apparatuses (110 and 201). Moreover, with regard to an individual photoacoustic imaging apparatus (202) which is non-operating due to, for example, discard or malfunction, an apparatus information database 205 is previously migrated to the medical information database 109. This enables reading out meta-information even about an image previously captured by the individual photoacoustic imaging apparatus 202. The target image readout unit 101 and the reference image readout unit 102 select appropriate individual photoacoustic imaging apparatuses based on apparatus identification information including in meta-information, and read out specific meta-information about each individual photoacoustic imaging apparatus. An individual apparatus in the present specification includes a photoacoustic imaging apparatus which is probable to be different in apparatus condition concerning an image capturing condition, and typically includes a photoacoustic imaging apparatus to which a serial number and a model number are specifically assigned. Such an individual apparatus can also be identified by, besides a serial number, for example, a maintenance record usable for tracing a history or a version number or revision number (revision edition) of implemented software or firmware for photoacoustic measurement.

In the present exemplary embodiment, as specific meta-information, a point image distribution function (point spread function (PSF)) of images is stored in an apparatus information database 205. The reason for this is described as follows.

The photoacoustic imaging apparatus has difficulty in measuring the entire circumference 2π ([rad] (or the entire solid angle 4π ([sr]) around a subject, unlike X-ray computed tomography (CT) and magnetic resonance imaging (MRI). This is because, while, in a photoacoustic imaging apparatus, it is necessary to fill a space between a subject and an acoustic wave detector with an acoustic matching agent, it is difficult to fill the entire circumference of the subject with the acoustic matching agent or it is difficult to locate the acoustic wave detector in such a way as to surround the entire circumference of the subject. Therefore, information obtained by image capturing suffers a loss with respect to an unmeasurable direction, so that the position dependence of resolution is larger than that of, for example, X-ray CT. The position dependence of resolution varies according to, for example, the size or location of an acoustic wave detector or the sensitivity difference between acoustic wave detectors, and, therefore, serves as specific meta-information for each individual photoacoustic imaging apparatus. Moreover, the resolution is generally expressed as a PSF. The PSF has a data structure of image data or volume data, and is, therefore, large in data amount and is difficult to record for each captured image. Furthermore, to express the position dependence, it is necessary to retain different PSFs for respective positions. For the reasons stated above, PSFs are stored in the apparatus information database 205.

To reduce the data amount, it is favorable that one PSF is retained for each space in which the PSF is approximately the same. The PSF is previously acquired by image capturing of a phantom in which point sound sources are located at a plurality of reference positions or by a simulation that is based on apparatus specifications.

The modification unit 103 performs modification processing on at least one of a target image and a reference image according to the processing methods concerning medical images described below, thus bringing the image qualities of the two images close to each other. The modification unit 103 is typically a program which is stored in the storage device 106 and which is read out and executed by the calculation device 100 at the time of execution of processing concerning medical images, and operates as a part of the calculation device 100. Modification is performed with use of meta-information read out by the target image readout unit 101 and the reference image readout unit 102. The modification unit 103 is equivalent to a modification unit in the present disclosure.

The display device 104 displays the target image and the reference image the image qualities of which have been brought close to each other by the modification unit 103. The user can view and compare the two images with each other via the display device 104, thus performing reading for interpretation and giving a diagnosis. Moreover, it is favorable that the display device 104 includes an interface for receiving, for example, selection of a target image targeted for diagnosis, selection of a reference image, and an instruction for parameters of modification processing. The interface to be used includes, for example, a graphical user interface (GUI) or a character user interface (CUI). Typically, for example, a liquid crystal display is used, but another type of display, such as a plasma display, an organic light emitting display (OLED), a field-emission display (FED), or a cathode-ray tube (CRT), can be used. The display device 104 is equivalent to a display unit in the present disclosure.

The user can use the input device 105 to perform, for example, selection of a target image targeted for diagnosis, selection of a reference image, and an instruction for parameters of modification processing. The input device 105 to be used includes, for example, a mouse, a keyboard, a trackball, and a touch panel. It is favorable that the input device 105 interlocks with the interface of the display device 104, thus facilitating issuance of an instruction to the apparatus.

The storage device 106 retains a program in which the processing method concerning medical images described below is described. For example, the storage device 106 retains a program equivalent to, for example, the target image readout unit 101, the reference image readout unit 102, and the modification unit 103. Moreover, the storage device 106 can be used to temporarily store, for example, a target image, a reference image, and meta-information read out from the medical information database 109. The storage device 106 to be employed includes, for example, a hard disk, an optical disc, a magneto-optical disc, an electrically erasable programmable read-only memory (EEPROM), or a flash memory.

The communication device 107 connects the processing apparatus 112 concerning medical images according to the present exemplary embodiment to the communication network 111. This enables reading out, for example, a target image, a reference image, and meta-information from the medical information database 109 or the photoacoustic imaging apparatus 110 or 201 connected to the communication network 111 to the processing apparatus 112 concerning medical images. The communication device 107 is typically, for example, a wired local area network (LAN) router, a wireless LAN router, or a modem.

The bus 108 connects the processing apparatus 112 concerning medical images according to the present exemplary embodiment to the medical information database 109 and the photoacoustic imaging apparatus 110, thus enabling exchange of information with each other.

The medical information database 109 stores a medical image and meta-information accompanied thereby acquired by the photoacoustic imaging apparatus 110. The stored medical image and meta-information are transferred to the processing apparatus 112 concerning medical images via the communication network 111, and are then used as a target image, a reference image, and their meta-information. Furthermore, it is favorable that the medical information database 109 is able to store electronic medical record information related to images. The medical information database 109 is typically, for example, a DICOM server, an electronic medical record server, a general-purpose data server, or a picture archiving and communication system (PACS) obtained by integrating them.

The photoacoustic imaging apparatus 110 captures an image of a subject by photoacoustic imaging and outputs the captured image as a medical image. The photoacoustic imaging apparatus 110 typically includes a light source which irradiates a subject with electromagnetic waves including light and an acoustic wave detector which receives acoustic waves generated inside the subject. The photoacoustic imaging apparatus 110 typically further includes a data acquisition system (DAS) which converts the received acoustic waves into a signal and an image reconstruction unit which reconstructs a subject image with use of the received acoustic waves. The DAS can be generally configured with an electrical circuit including an A/D converter. The image reconstruction unit can be generally configured with a workstation including a CPU or a GPU and a memory or a hard disk. It is favorable that the image reconstruction unit is able to easily record meta-information and to output a subject image in a DICOM format which is high in affinity for a DICOM server commonly used as a medical information database.

The communication network 111 is a communication pathway which interconnects the processing apparatus 112 concerning medical images according to the present exemplary embodiment, the medical information database 109, and the photoacoustic imaging apparatus 110, thus enabling exchange of information with each other. The communication network 111 is typically, for example, a wide area network (WAN), a local area network (LAN), a telephone line, an Internet line, or an intra-hospital network. Moreover, the communication network 111 can be built out of any configuration, such as a wired, wireless, or their mixed configuration.

### <Processing Method Concerning Medical Images>

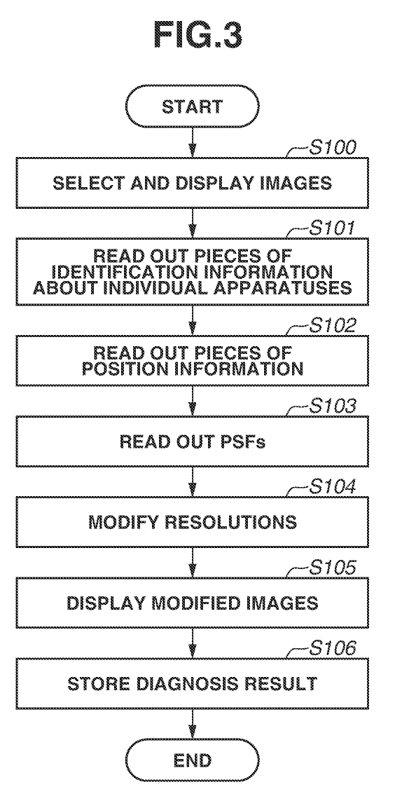

Next, each process in the processing method concerning medical images according to the present exemplary embodiment is described with reference to FIG. 3. Furthermore, each process is performed by the calculation device 100 controlling an operation of each constituent element of the processing apparatus 112 concerning medical images.

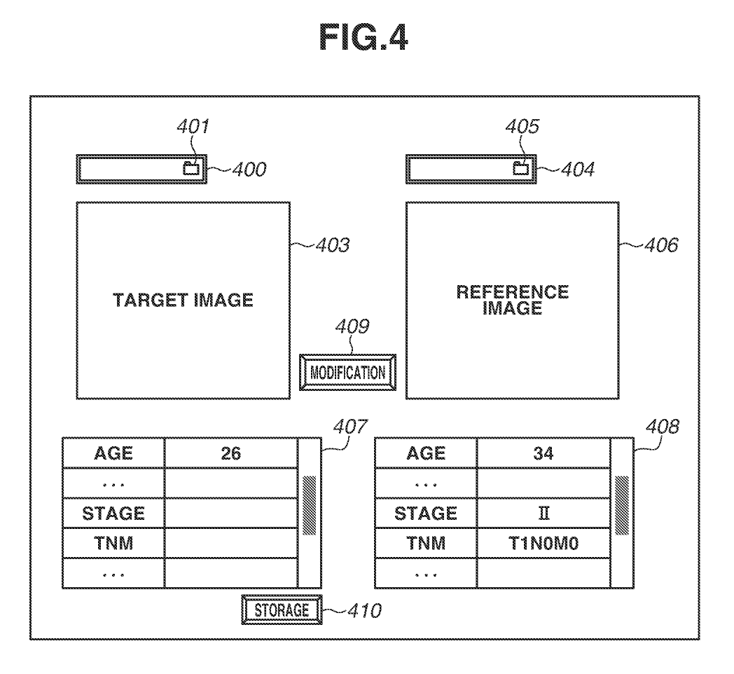

In step S100 (a process of selecting and displaying a target image and a reference image), the calculation device 100 reads out a target image targeted for diagnosis and a reference image targeted for comparison, selected by the user, from the medical information database 109 and displays the read-out target image and the read-out reference image on the display device 104. FIG. 4 illustrates an example of a GUI which is displayed on the display device 104. When the user enters a target image path into a target image path entry portion 400 or the user clicks a list display button 401 for target images and selects a target image is displayed on an image display portion 403. Moreover, when the user enters a reference image path into a reference image path entry portion 404 or the user clicks a list display button 405 for reference images and selects a reference image from a list of reference images, a reference image is displayed on an image display portion 406. At this time, pieces of meta-information about the read-out target image and the read-out reference image can be displayed on meta-information display portions 407 and 408, respectively. The user can select a reference image appropriate for the read-out target image based on the displayed meta-information. For example, when the user selects a reference image closer to the read-out target image with respect to age based on the age of a subject included in the meta-information, a characteristic difference between two images dependent on subjects can be reduced in advance.

In step S101 (a process of reading out apparatus identification information), the calculation device 100 reads out apparatus identification information for identifying an individual apparatus, which is meta-information, with respect to each of the read-out target image and the read-out reference image from the apparatus information database 205 included in the medical information database 109. The apparatus identification information is identification information indicating an individual photoacoustic imaging apparatus which was used to capture each image. In the present exemplary embodiment, the apparatus identification information is expressed by an integer n (n=0, 1, . . . ). Apparatus identification information about the target image is assumed to be nᵢ, and apparatus identification information about the reference image is assumed to be nᵣ.

In step S102 (a process of reading out pieces of position information about a target image and a reference image), the calculation device 100 reads out position information 210a about a corresponding image included in the meta-information 200 with respect to each of the read-out target image and the read-out reference image from the medical information database 109. The position of the read-out target image is assumed to be xᵢ, and the position of the read-out reference image is assumed to be xᵣ. The position information 210a is meta-information uniquely specified for each of the read-out target image and the read-out reference image, and is a vector quantity indicating the image capturing position in a subject an image of which was captured as the corresponding image.

In step S103 (a process of reading out PSFs of a target image and a reference image), the calculation device 100 reads out, from the apparatus information database 205, PSFs at the respective positions of the individual apparatuses which respectively captured the read-out target image and the read-out reference image, based on the apparatus identification information read out in step S101 and the position information 210a read out in step S102. When a PSF which depends on the apparatus identification information n and the position x is assumed to be hₙ(x, u), the PSF of the read-out target image is expressed as hₘ(xᵢ, u) and the PSF of the read-out reference image is expressed as hₙᵣ(xᵣ, u).

In step S104 (a process of modifying resolutions), the calculation device 100 modifies at least one of resolutions of the read-out target image and the read-out reference image with use of the PSFs read out in step S103 by the modification unit 103 included in the calculation device 100, thus bringing the image qualities of the two images close to each other. This step is performed when the user presses a modification button 409 after finalizing the target image and the reference image in step S100.

Processing which is performed to bring the image qualities of the two images close to each other by excluding the influence of a PSF from both of the read-out target image and the read-out reference image is described. When the read-out target image is denoted by gᵢ(u), the read-out reference image is denoted by gᵣ(u), a target image not yet subjected to a PSF is denoted by gᵢ⁰(u), and a reference image not yet subjected to a PSF is denoted by gᵣ⁰(u), the read-out target image and the read-out reference image are expressed as follows.

gᵢ(u)=∫gᵢ⁰(w)hₙ_{ᵢ}(xᵢ,u−w)dw (1)

gᵣ(u)=∫gᵣ⁰(w)hₙ_{ᵣ}(xᵣ,u−w)dw (2)

When the formulae (1) and (2) are converted into frequency spaces, the following formulae are obtained.

Gᵢ(f)=Gᵢ⁰(f)Hₙ_{ᵢ}(xᵢ,f) (3)

Gᵣ(f)=Gᵣ⁰(f)Hₙ_{ᵣ}(xᵣ,f) (4)

In the formulae (3) and (4), f denotes frequency and an uppercase function denotes a frequency space representation of a lowercase function. The modification of solutions is performed by the following formulae (5) and (6), which are obtained by transforming the formulae (3) and (4).

$$\begin{matrix}{G}_{i}^{0} ( f ) = \frac{{G}_{i} ( f )}{{H}_{{n}_{i}} ( {x}_{i} , f )} & ( 5 ) \\ {G}_{r}^{0} ( f ) = \frac{{G}_{r} ( f )}{{H}_{{n}_{r}} ( {x}_{r} , f )} & ( 6 )\end{matrix}$$

Actually, since a frequency component the absolute value of which is zero or a noise is included, it is favorable that Wiener filters expressed as the following formulae (7) and (8) are used.

In this regard, in the formulae (7) and (8), the overline (⁻) represents the complex conjugate, and the tilde (˜) represents the approximate solution. σ is a sufficiently small value relative to |H|², and is generally a value corresponding to several percent of the maximum value of |H|².

$$\begin{matrix}{\tilde{G}}_{i}^{0} ( f ) = \frac{{\overline{H}}_{{n}_{i}} ( {x}_{i} , f )}{{\vert {H}_{{n}_{i}} ( {x}_{i} , f ) \vert}^{2} + σ} {G}_{i} ( f ) & ( 7 ) \\ {\tilde{G}}_{r}^{0} ( f ) = \frac{{\overline{H}}_{{n}_{r}} ( {x}_{r} , f )}{{\vert {H}_{{n}_{r}} ( {x}_{r} , f ) \vert}^{2} + σ} {G}_{r} ( f ) & ( 8 )\end{matrix}$$

When the thus-obtained

and

\tilde{G}ᵣ⁰(f)

are inversely transformed into real spaces, a modified target image gᵢ′(u) and a modified reference image gᵣ′(u) are obtained.

Furthermore, the transformation into a frequency space can be performed by using an appropriate method according to the location of an acoustic wave detector of the photoacoustic imaging apparatus 110. For example, in a case where the acoustic wave detector is located in a plane surface, since a PSF tends to spread in a planer direction, Fourier transform is favorable. In a case where the acoustic wave detector is located in a spherical surface, since a PSF tends to spread in a spherical shell direction, transform using spherical surface harmonics is favorable.

A repeated optimization calculation can be used as a method of estimating gᵢ′(u) (gᵣ′(u)) from the formula (1) (formula (2)). In other words, gᵢ⁰(u) (gᵣ⁰(u)) in the formula (1) (formula (2)) is replaced by gᵢ′(u) (gᵣ′(u)), and gᵢ′(u) (gᵣ′(u)) whose square error with gᵢ(u) (gᵣ(u)) becomes minimum is set as a modified image. In this case, it is favorable to reduce inappropriateness of a repeated calculation by performing optimization with addition of an appropriate regularization term.

While, up to here, the case of excluding the influence of a PSF from both of the read-out target image and the read-out reference image has been described, the PSF of the read-out target image can be converted into the PSF of the read-out reference image to bring the image qualities thereof close to each other. In this case, the following formula (9) is used.

$${G}_{i}^{′} ( f ) = ( \frac{{H}_{{n}_{i}} ( {x}_{i} , f )}{{\vert {H}_{{n}_{i}} ( {x}_{i} , f ) \vert}^{2} + σ} {G}_{i} ( f ) ) {H}_{{n}_{r}} ( {x}_{r} , f ) \qquad ( 9 )$$

The inside of parentheses in the right-hand side of the formula (9) is a frequency representation of an image obtained by excluding the influence of a PSF from the read-out target image. This representation is multiplied by Hₙᵣ, which is a frequency representation of the PSF of the read-out reference image, so that a frequency representation Gi′ of the modified target image is obtained. A modified image gᵢ′(u) is obtained by inversely converting Gi′ (f). Similarly, the PSF of the read-out reference image can be converted into the PSF of the read-out target image to bring the image qualities thereof close to each other.

In the above-described way, a resolution difference between the read-out target image and the read-out reference image can be suppressed. Furthermore, according to the formulae (7), (8), and (9), not only a resolution difference but also a contrast difference between the read-out target image and the read-out reference image can be suppressed.

In step S105 (a process of displaying modified images), the calculation device 100 displays at least one of the modified target image gᵢ′ (u) and the modified reference image gᵣ′(u), obtained by modification in step S104, at the image display portions 403 and 406, respectively. In a case where only one of the two images is subjected to modification in step S104, the image not subjected to modification is kept in a state of being displayed in step S100. Since the influence of a difference in PSF caused by a difference in individual apparatus and a difference in location has been suppressed in step S104, the displayed images are images appropriate for the user to make a diagnosis.

In step S106 (a process of storing a diagnosis result), the calculation device 100 stores, in the medical information database 109, a diagnosis result determined by the user based on the modified target image and the modified reference image displayed in step S105. When the user enters a diagnosis result into a diagnosis item of the meta-information display portion 407 and then presses a storage button 410, the diagnosis result is stored in the medical information database 109.

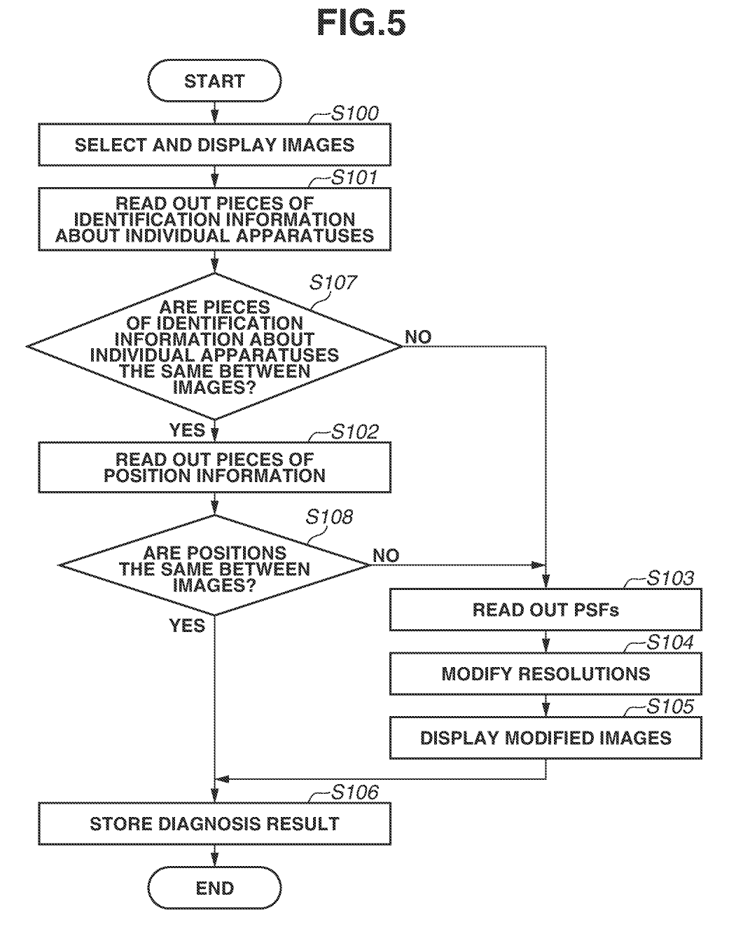

While, in the above description, a configuration in which modification is performed on at least one of two images to be compared with each other has been described, a configuration in which an image not subjected to modification that is based on meta-information is included in the read-out reference image or the read-out target image can be employed. This enables reducing, for example, a calculation time for modification processing, thus shortening a diagnosis time. In step S107 and step S108 illustrated in FIG. 5, the calculation device 100 determines whether the apparatus which was used to capture the target image and the apparatus which was used to capture the reference image are the same and whether the positions of the respective two images are in approximately the same region in the PSF. If the results of two determinations are true (YES in step S107 and YES in step S108), the calculation device 100 does not perform steps S103, S104, and S105.

In the present exemplary embodiment, since not only meta-information recorded with each of a target image and a reference image but also meta-information about an apparatus which was used to capture an image is used, a difference in at least one of resolution and contrast caused by a difference between apparatuses which were used to capture images and a difference in position between images can be reduced. This enables bringing the image qualities of the read-out target image and the read-out reference image close to each other with a high degree of accuracy, thus increasing diagnostic accuracy.

Furthermore, meta-information to be read out in each process in the present exemplary embodiment only needs to be read out from a storage medium in which the meta-information is stored, and a configuration in which the meta-information is read out from other than the databases illustrated as an example in the present exemplary embodiment is also included as one configuration of the present disclosure.

A second exemplary embodiment is configured to modify a difference between images caused by a difference in wavelength between measurement rays of light with which to irradiate a subject to capture a target image and a reference image. Furthermore, in principle, the same constituent elements as those in the first exemplary embodiment are assigned the respective same reference characters, and the description thereof is not repeated.

### <Configuration of a Processing Apparatus Concerning Medical Images>

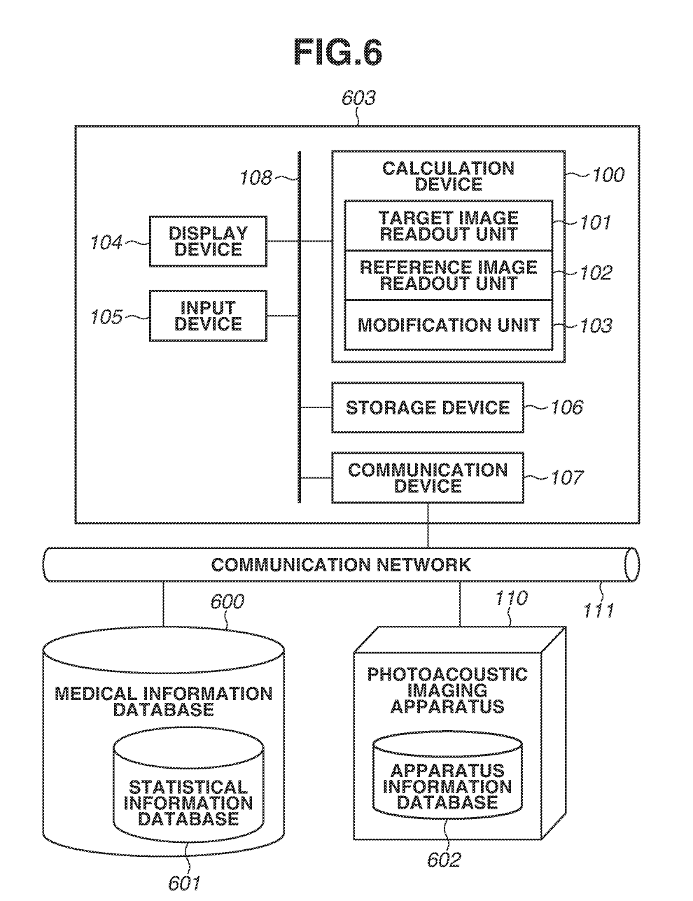

FIG. 6 is a schematic diagram of a processing apparatus 603 concerning medical images according to the second exemplary embodiment. Hereinafter, each constituent element of the processing apparatus 603 is described. While, basically, the processing apparatus 603 has a configuration equivalent to that in the first exemplary embodiment, the medical information database 109 is replaced by a medical information database 600 including a statistical information database 601, and the apparatus information database 203 is replaced by an apparatus information database 602.

Hereinafter, details of each constituent element of the processing apparatus 603 concerning medical images according to the second exemplary embodiment are described.

The medical information database 600 is basically equivalent to that in the first exemplary embodiment, but differs from the medical information database 109 in the first exemplary embodiment in that the medical information database 600 includes the statistical information database 601, which stores statistical values calculated from the stored group of images and the stored group of pieces of meta-information. In the statistical information database 601, if a new image or new meta-information is requested to be added, the new image or the new meta-information is added to be written as needed, so that a group of pieces of data is updated. The statistical values can be dynamically generated in response to an external request. In the second exemplary embodiment, statistical values of background optical coefficients described below relative to wavelengths are stored in the statistical information database 601.

The apparatus information database 602 differs from the apparatus information database 203 in the first exemplary embodiment in that the apparatus information database 602 retains spectral of light energy with which to irradiate a subject as meta-information specific for the photoacoustic imaging apparatus 110. The spectral of light energy with which to irradiate a subject are also called irradiation light spectral, and are retained in the apparatus information database 602 as apparatus information which relies on an irradiation optical system (not illustrated) included in the photoacoustic imaging apparatus 110. The irradiation light spectral are determined by a light source included in the irradiation optical system and optical elements located in a transmission path leading from the light source to a light exit portion. The above-mentioned statistical values of background optical coefficients and the irradiation light spectral are read out from at least one of the target image readout unit 101 and the reference image readout unit 102.

### <Processing Method Concerning Medical Images>

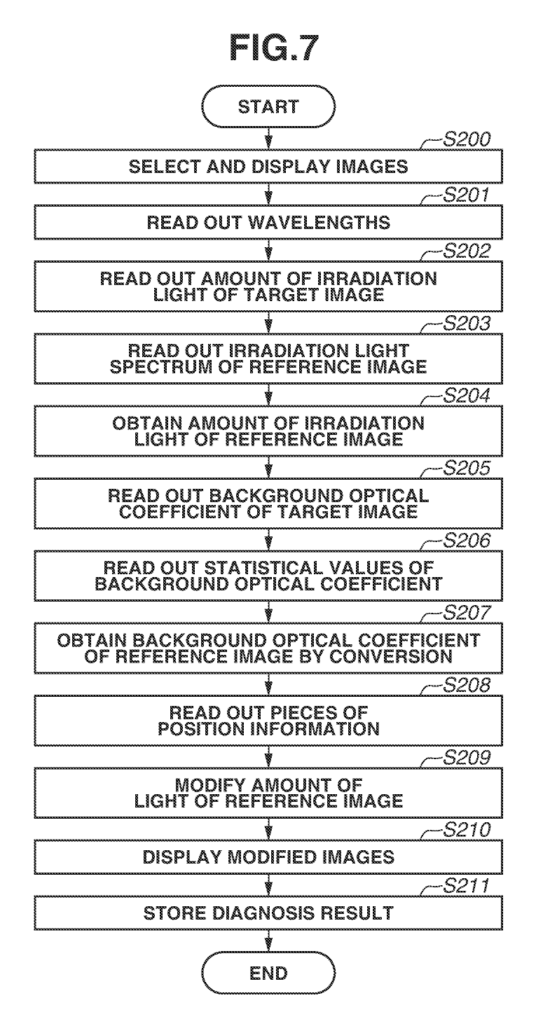

Next, each process in the processing method concerning medical images according to the second exemplary embodiment is described with reference to FIG. 7. Furthermore, each process is performed by the calculation device 100 controlling an operation of each constituent element of the processing apparatus 603 concerning medical images.

In step S200 (a process of selecting and displaying a target image and a reference image), which is basically equivalent to step S100 in the first exemplary embodiment, the calculation device 100 further displays light-source wavelengths of rays of light used to capture the read-out target image and the read-out reference image at the meta-information display portions 407 and 408, respectively. The user can select a reference image as close to the target image as possible with respect to wavelengths, a characteristic difference between two images dependent on light-source wavelengths used at the time of image capturing can be reduced in advance.

In the second exemplary embodiment, the target image and the reference image are assumed to be initial sound pressure images. While, in the medical information database 600, the amount of irradiation light used to irradiate a subject at the time of image capturing and the background optical coefficients described below are recorded as meta-information associated with the read-out target image, a case in which those are not recorded with respect to the read-out reference image is assumed. For example, in a case where meta-information which was not recorded at the time of capturing a reference image has been newly recorded at the time of capturing a target image, such a situation occurs.

In step S201 (a process of reading out measurement wavelengths for the target image and the reference image), the calculation device 100 reads out wavelengths used at the time of measurement respectively associated with the read-out target image and the read-out reference image as meta-information from the medical information database 600. The measurement wavelength for the read-out target image is assumed to be λᵢ, and the measurement wavelength for the read-out reference image is assumed to be λᵣ. Such measurement wavelengths are wavelengths representative of image capturing conditions for photoacoustic measurement corresponding to the respective images, and are included in spectral of irradiation light to be radiated from a light exit unit (not illustrated) onto a subject.

In step S202 (a process of reading out the amount of irradiation light of the target image), the calculation device 100 reads out, from the medical information database 600, the amount of irradiation light serving as meta-information about the read-out target image based on the measurement wavelength λᵢ read out in step S201. The amount of irradiation light for the read-out target image is assumed to be Eᵢ(λᵢ). The amount of irradiation light is equivalent to an output intensity obtained by integrating all of the light fluxes radiated from the light exit unit (not illustrated) onto a region of interest in the measurement wavelength λᵢ.

In step S203 (a process of reading out an irradiation light spectrum of the read-out reference image), the calculation device 100 reads out an irradiation light spectrum of the read-out reference image from the apparatus information database 602.

In step S204 (a process of obtaining the amount of irradiation light of the read-out reference image), the calculation device 100 obtains the amount of irradiation light Eᵣ(λᵣ) in the measurement wavelength λᵣ of the read-out reference image from the irradiation light spectrum acquired in step S203.

In step S205 (a process of reading out a background optical coefficient of the target image), the calculation device 100 reads out a background optical coefficient of the target image serving as meta-information from the medical information database 600. The background optical coefficient is one of parameters describing the amount of light inside an optical absorption scattering body such as a biological object, and is described as a set of a background absorption coefficient (μₐ_{_}_{b}), which indicates how easily light is absorbed, and a background scattering coefficient (μ′ₛ_{_}_{b}), which indicates how easily light is scattered. Furthermore, typically, an average value for the entire subject serving as an image capturing target is employed as the background optical coefficient. The amount of light φ(x) at the position x in the subject is expressed by the following diffusion equation (10) including the background optical coefficient. q(x) is a source term which is proportional to the amount of irradiation light.

$$\frac{1}{3 {μ}_{s \_ b}^{′}} ∇ \cdot [ ∇ φ ( x ) ] - {μ}_{a \_ b} \cdot φ ( x ) + q ( x ) = 0 \qquad ( 10 )$$

The background optical coefficient differs according to subjects and measurement wavelengths, and is, therefore, recorded with a target image as meta-information. Furthermore, the function of measuring the background optical coefficient is included in the photoacoustic imaging apparatus 110. For example, that function can be implemented as a measurement device using a time-resolved measurement method or a phase modulation measurement method or a program for estimating the background optical coefficient based on an image captured by the photoacoustic imaging apparatus 110.

The background optical coefficient of the target image is assumed to be (μₐ_{_}_{b}(λᵢ), μ′ₛ_{_}_{b}(λᵢ)).

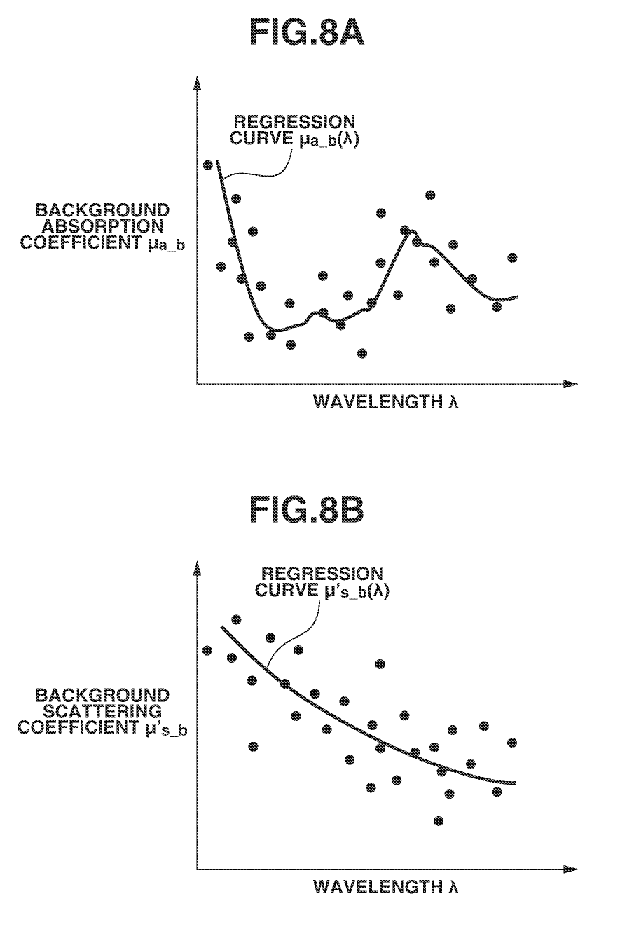

In step S206 (a process of reading out statistical values of the background optical coefficient), the calculation device 100 reads out statistical values of the background optical coefficient from the statistical information database 601. FIGS. 8A and 8B illustrate statistical values of the background optical coefficient. A filled circle indicates a background optical coefficient read from meta-information about each image stored in the medical information database 600, and a solid curved line indicates a regression curve obtained from a plurality of read background optical coefficients.

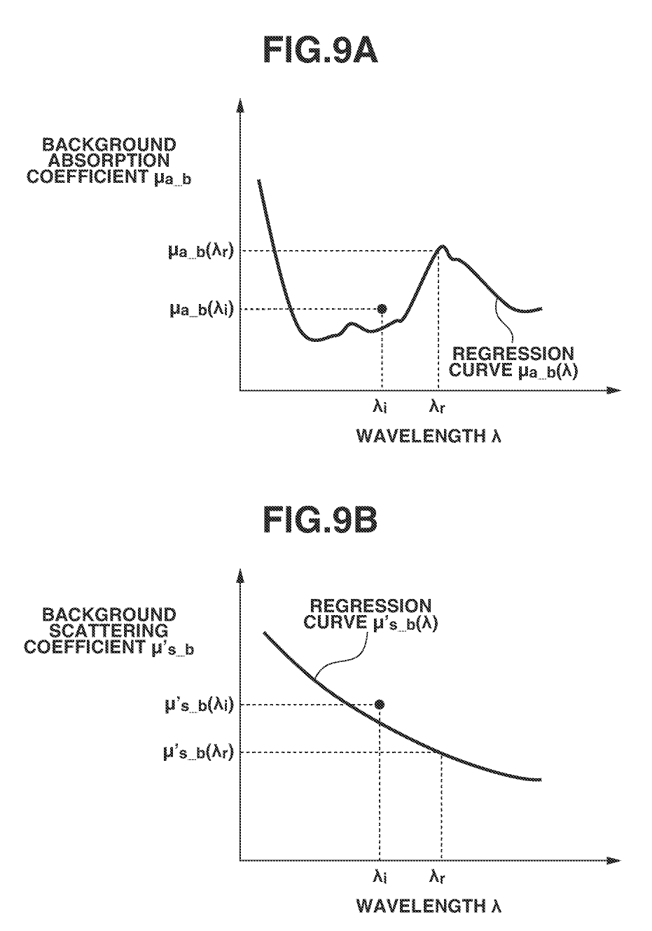

In step S207 (a process of obtaining the background optical coefficient of the read-out reference image), the calculation device 100 obtains the background optical coefficient (μₐ_{_}_{b}(λᵣ), λ′ₛ_{_}_{b}(λᵣ)) in the measurement wavelength λᵣ of the read-out reference image with use of the statistical values (regression curve) of the background optical coefficient acquired in step S206. FIGS. 9A and 9B illustrate a relationship between the background optical coefficients obtained in steps S205 and S207.

Step S208 (a process of reading out pieces of position information about the read-out target image and the read-out reference image) is equivalent to step S102.

In step S209 (a process of modifying the amount of light of the read-out reference image), the calculation device 100 converts the amount of light of the read-out reference image into the amount of light of the read-out target image, thus modifying a difference between the images caused by a difference in measurement wavelength between the two images.

The calculation device 100 assigns the background optical coefficient (μₐ_{_}_{b}(λᵢ), μ′ₛ_{_}_{b}(λᵢ)) and the amount of irradiation light Eᵢ(λᵢ) of the read-out target image, acquired in the processes up to here, to the formula (10), thus obtaining the following formula (11). During assignment of Eᵢ(λᵢ), the amount of irradiation light Eᵢ(λᵢ) is subjected to appropriate conversion and is then reflected in the source term q(x), so that qᵢ(x) is obtained.

$$\frac{1}{3 {μ}_{s \_ b}^{′}} ∇ \cdot [ ∇ φ ( x ) ] - {μ}_{a \_ b} ( {λ}_{i} ) \cdot φ ( x ) + {q}_{i} ( x ) = 0 \qquad ( 11 )$$

When the formula (11) is solved and x=xᵢ is assigned, the amount of light at the position xᵢ of the read-out target image, φ=φᵢ(xᵢ), is obtained. The method of solving the formula (11) can include another method, such as analytical solution or numerical solution.

With regard to the read-out reference image, the amount of light at the position xᵢ of the read-out target image, φ=φᵣ(xᵣ), is also obtained in a way similar to that in the case of the read-out target image.

Next, the amount of light of the read-out reference image is converted into the amount of light of the read-out target image. Since an initial sound pressure P in photoacoustic imaging is proportional to the amount of light, when the read-out reference image is assumed to be gᵣ(u), a modified reference image gᵣ′(u) can be obtained by the following formula (12).

$${g}_{r}^{′} ( x ) = \frac{{φ}_{i} ( {x}_{i} )}{{φ}_{r} ( {x}_{r} )} \cdot {g}_{r} ( u ) \qquad ( 12 )$$

In step S210 (a process of displaying modified images), the calculation device 100 displays the read-out target image and the modified reference image obtained in step S209 at the image display portions 403 and 406, respectively. Since the influence of a difference in amount of light caused by a difference in measurement wavelength has been suppressed in step S209, the displayed images are images appropriate for the user to make a diagnosis.

Step S211 (a process of storing a diagnosis result) is equivalent to step S106.

While, in the above description, a case in which the read-out reference image is modified so that the image quality thereof is brought close to the image quality of the read-out target image has been described, the image quality of the read-out target image can be brought close to the image quality of the read-out reference image. Moreover, while the diffusion equation (10) is used as a formula for calculating the amount of light, the method of calculating the amount of light of an optical absorption scattering body can include a general method, such as a Monte Carlo method or a transport equation.

Furthermore, while, in the present exemplary embodiment, both the amount of irradiation light and the background optical coefficient are modified, the advantageous effect of the present exemplary embodiment can be attained even by modification of only either one.

Furthermore, while, in the present exemplary embodiment, the amount of irradiation light is modified with respect to a corresponding image, since the amount of irradiation light is modified based on the position information, it can also be said that a distribution of the amount of light of a corresponding image is modified.

In the present exemplary embodiment, using not only meta-information recorded with the read-out target image and the read-out reference image but also meta-information about apparatuses used to capture images and statistical information obtained from a plurality of medical images enables reducing a difference in image quality (luminance value) caused by a difference in measurement wavelength. This enables bringing the image qualities of the read-out target image and the read-out reference image close to each other with a high degree of accuracy, thus increasing diagnostic accuracy.

Furthermore, with regard to meta-information to be read out in each process in the present exemplary embodiment, as in the first exemplary embodiment, a configuration in which the meta-information is read out from other than the databases illustrated as an example in the present exemplary embodiment is also included as one configuration of the present disclosure.

Moreover, the first exemplary embodiment and the second exemplary embodiment can be combined. This enables bringing the image qualities of the read-out target image and the read-out reference image close to each other with a higher degree of accuracy.

Details of the present disclosure have been described above with reference to specific exemplary embodiments. However, the present invention is not limited to the above specific exemplary embodiments, but can be modified or altered in exemplary embodiment within the range not departing from the technical idea of the present disclosure.

As described above, according to the present disclosure, images can be provided with a small amount of calculation time while the image qualities in photoacoustic imaging or ultrasonic echo imaging are improved.

While the present disclosure has been described with reference to exemplary embodiments, it is to be understood that the invention is not limited to the disclosed exemplary embodiments. The scope of the following claims is to be accorded the broadest interpretation so as to encompass all such modifications and equivalent structures and functions.

This application claims the benefit of Japanese Patent Application No. 2017-176982, filed Sep. 14, 2017, which is hereby incorporated by reference herein in its entirety.

What is claimed is:

1. A processing apparatus concerning medical images comprising:
a reference image readout unit configured to read out a reference image acquired by photoacoustic imaging;
a target image readout unit configured to read out a target image for diagnostics acquired by photoacoustic imaging; and
a calculation unit configured to modify at least one of a read-out reference image and a read-out target image,
wherein the reference image and the target image are read out while being associated with meta-information, and
wherein the calculation unit modifies at least one of the read-out reference image and the read-out target image based on the meta-information.

2. The processing apparatus concerning medical images according to claim 1, wherein the meta-information includes information concerning an image capturing condition with which photoacoustic image capturing of a corresponding one of the images was performed.

3. The processing apparatus concerning medical images according to claim 1, wherein the meta-information includes information for identifying a photoacoustic imaging apparatus which was used to capture a corresponding one of the images.

4. The processing apparatus concerning medical images according to claim 1, wherein the meta-information includes position information indicating an image capturing position inside a subject a corresponding one of the images of which was captured.

5. The processing apparatus concerning medical images according to claim 1, wherein the meta-information includes information about a point spread function of a corresponding one of the images.

6. The processing apparatus concerning medical images according to claim 1, wherein the meta-information includes an amount of light with which a subject was irradiated to capture a corresponding one of the images.

7. The processing apparatus concerning medical images according to claim 1, wherein the meta-information includes a distribution of amount of light with which a subject was irradiated to capture a corresponding one of the images.

8. The processing apparatus concerning medical images according to claim 1, wherein the meta-information includes a background optical coefficient of a subject serving as an image capturing target of a corresponding one of the images.

9. The processing apparatus concerning medical images according to claim 1, wherein the meta-information includes a wavelength of light with which a subject was irradiated to capture a corresponding one of the images.

10. The processing apparatus concerning medical images according to claim 1, wherein the meta-information is a statistical value of a background optical coefficient of a subject a corresponding one of the images of which was captured.

11. The processing apparatus concerning medical images according to claim 1, wherein the meta-information is stored while being associated with a corresponding one of the images.

12. The processing apparatus concerning medical images according to claim 1, wherein the meta-information is stored in photoacoustic imaging apparatuses which were used to capture a corresponding plurality of images of the images while being associated with the plurality of images in common.

13. The processing apparatus concerning medical images according to claim 1, wherein at least one of the target image readout unit and the reference image readout unit reads out, based on information for identifying an apparatus which was used to capture a corresponding one of the images, the meta-information from the apparatus which was used to capture the corresponding one of the images.

14. The processing apparatus concerning medical images according to claim 1, wherein at least one of the target image readout unit and the reference image readout unit reads out, based on a wavelength of light with which a subject was irradiated to capture a corresponding one of the images, the meta-information from a database storing medical images.

15. The processing apparatus concerning medical images according to claim 1, wherein the calculation unit modifies at least one of a resolution and a contrast of at least one of the read-out target image and the read-out reference image based on the meta-information.

16. The processing apparatus concerning medical images according to claim 1,
wherein at least one of the target image readout unit and the reference image readout unit acquires a wavelength of irradiation light which was used to capture at least one of the read-out target image and the read-out reference image based on the meta-information, and
wherein the calculation unit calculates a background optical coefficient based on the acquired wavelength and modifies a luminance value of the at least one.

17. The processing apparatus concerning medical images according to claim 1, further comprising a display unit configured to display the read-out target image and the read-out reference image.

18. A processing method concerning medical images comprising:
reading out a reference image acquired by photoacoustic imaging;
reading out a target image for diagnostics acquired by photoacoustic imaging; and
modifying at least one of a read-out reference image and a read-out target image,
wherein the reference image and the target image are read out while being associated with meta-information, and
wherein the at least one of the read-out reference image the read-out target image is modified based on the meta-information.

19. The processing method concerning medical images according to claim 18, wherein the meta-information includes information concerning an image capturing condition with which photoacoustic image capturing of a corresponding one of the images was performed.

20. The processing method concerning medical images according to claim 18, wherein the meta-information includes information for identifying a photoacoustic imaging apparatus which was used to capture a corresponding one of the images.

21. The processing method concerning medical images according to claim 18, wherein the meta-information includes position information indicating an image capturing position inside a subject a corresponding one of the images of which was captured.

22. The processing method concerning medical images according to claim 18, wherein the meta-information includes information about a point spread function of a corresponding one of the images.

23. The processing method concerning medical images according to claim 18, wherein the meta-information includes an amount of light with which a subject was irradiated to capture a corresponding one of the images.

24. The processing method concerning medical images according to claim 18, wherein the meta-information includes a distribution of amount of light with which a subject was irradiated to capture a corresponding one of the images.

25. The processing method concerning medical images according to claim 18, wherein the meta-information includes a background optical coefficient of a subject serving as an image capturing target of a corresponding one of the images.

26. The processing method concerning medical images according to claim 18, wherein the meta-information includes a wavelength of light with which a subject was irradiated to capture a corresponding one of the images.

27. The processing method concerning medical images according to claim 18, wherein the meta-information is a statistical value of a background optical coefficient of a subject a corresponding one of the images of which was captured.

28. The processing method concerning medical images according to claim 18, wherein the meta-information is stored while being associated with a corresponding one of the images.
